(19) United States
(12) Patent Application Publication
(10) Pub. No.: US 2011/0137176 A1
(43) Pub. Date: Jun. 9, 2011

(54) ULTRASOUND TRANSDUCER FINGER PROBE

(75) Inventors: David G. MILLER, North Andover, MA (US); Michael E. PESZYNSKI, Newburyport, MA (US)

(73) Assignee: Koninklijke Philips Electronics N.V., Eindhoven (NL)

(21) Appl. No.: 12/785,657

(22) Filed: May 24, 2010

Related U.S. Application Data
(62) Division of application No. 10/965,508, filed on Oct. 14, 2004, now abandoned.
(60) Provisional application No. 60/512,997, filed on Oct. 21, 2003.

(51) Int. Cl. A61B 8/00

(52) U.S. Cl. 600/459

(57) ABSTRACT

Finger probe for use in ultrasonic imaging includes a housing and a matrix array arranged within the housing to produce ultrasound beams and which includes a plurality of independently-addressable transducer elements. A finger clip is coupled to the housing and arranged to accommodate an operator's finger. A control unit is coupled to the transducer elements to provide planar and volumetric scanning capabilities. A system and method for ultrasonically interrogating a patient's body part and for producing ultrasound images based on the interrogation using a finger-mounted ultrasound probe are also disclosed.

### CROSS REFERENCE TO RELATED CASES

Applicants claim the benefit of Provisional Application Ser. No. 60/512,997, filed 21 Oct. 2003.

The present invention relates generally to ultrasound transducer probes for use in interoperative ultrasound imaging and more particularly, to an ultrasound transducer probe that can be attached to a physician's finger for use in interoperative and intra-cavity ultrasound imaging applications.

The invention also relates to a system and method for ultrasonically interrogating a patient's body part and for producing ultrasound images based on the interrogation using a finger-mounted ultrasound probe.

Rotatable ultrasound transducer probes, such as transesophageal or TEE probes, are used for viewing planar ultrasound images of a patient's heart from inside of the patient's esophagus. The tip of the TEE probe houses a rotatable array element. Rotation of the array element causes a corresponding rotation of the image plane about an image axis. Once the TEE probe is inserted down the esophagus, rotation of the array element is controlled at a remote distance from the probe tip.

In contrast to TEE probes, in which rotation of the image plane is controlled at a remote distance from the probe tip, finger probes are attached to a physician's finger. The image plane orientation is then manually controlled by the movement of the physician's finger. Finger probes are well suited for internal imaging through the body cavities and in interoperative environments, such as during open heart surgery or vascular surgery.

Hanaoka et al., in U.S. Pat. No. 5,284,147, describe one type of finger probe which uses a stationary imaging element. While the image axis of the stationary imaging element may be readily aimed at the patient's body part to be viewed, rotation of the image plane about the image axis to obtain other critical views of the patient's body part is implemented by physically rotating the finger probe and its attached cable. Since the body cavities into which the finger probe is inserted are often small and space constrained, physical rotation of the finger probe is limited, which reduces viewing access to the patient's body parts.

Peszynski, in U.S. Pat. No. 5,598,846, describes another type of finger probe which uses a rotatable imaging array element to achieve rotation of the image plane. A finger clip provides attachment of the rotatable finger probe to a physician's finger which allows the finger to aim the imaging axis at a patient's body part. A foot switch or other remote control mechanism controls rotation of the image plane about an image axis for acquisition of various views of the patient's body.

The imaging capabilities of the prior art finger probes have not kept pace with technological advances and therefore a new finger probe with advanced imaging capabilities is desirable. Specifically, with the above-described finger probes, it is necessary to rotate the imaging element to obtain planar images from different views of the object being examined in the patient's body. Accordingly, the finger probe must include an imaging element which is designed to be rotated and associated structure to provide for rotation of the imaging element during an examination.

It would be advantageous to obtain volumetric, three-dimensional views of the object being examined without requiring rotation of an imaging element.

It is an object of the present invention to provide a new ultrasound finger probe which has advanced imaging capabilities in comparison to prior art ultrasonic finger probes.

It is another object of the present invention to provide a new ultrasound finger probe which does not require a rotatable imaging element or associated structure to provide for rotation of such an imaging element.

It is another object of the present invention to provide a new ultrasound finger probe which provides volumetric, three-dimensional views of the object being examined without requiring rotation of an imaging element.

It is yet another object of the present invention to provide a new and improved system and method for ultrasonically interrogating a patient's body part and for producing ultrasound images based on the interrogation using a finger-mounted ultrasound probe.

In order to achieve this object and others, a finger probe for use in ultrasonic imaging in accordance with the invention includes a housing, a matrix array arranged within the housing to produce ultrasound beams and including a plurality of independently-addressable transducer elements, a finger clip coupled to the housing and arranged to accommodate an operator's finger. By providing the independently-addressable transducer elements, it is possible to control the transducer elements to obtain different images of an object being examined, including planar and volumetric, three-dimensional images, without requiring any sort of rotation of the matrix array.

By providing a matrix array instead of the transducer arrays as in the prior art mentioned above, it is not necessary to provide structure to cause rotation of the matrix array, i.e., the matrix array is non-rotatable relative to the housing, since the same effect of rotation of the array element in the prior art is now being obtained electronically via control of the transducer elements.

The matrix array may comprise transducer elements bonded to an array backing and connected to an integrated circuit which is connected in turn to a circuit board. The transducer elements may be segmented into (or designated as) transmit sub-arrays and receive sub-arrays. Each transmit sub-array may be connected to a respective intra-group transmit pre-processor which is connected to a respective transmit beamformer channel.

Each receive sub-array may be connected to a respective intra-group receive pre-processor which is connected to a respective receive beamformer channel. Control of the sub-arrays is obtained by a control processor in a manner known in the art, for example, as disclosed in U.S. Pat. No. 6,572,547 incorporated by reference herein.

In a system and method for ultrasonically interrogating a patient's body part and for producing ultrasound images based on the interrogation in accordance with the invention, an ultrasound probe including a matrix array is arranged to produce an ultrasound beam and receive reflections of the beam by the patient's body part. A finger clip is coupled to the probe to enable attachment of the probe to an operator's finger. A display unit is coupled to the probe for displaying ultrasound images based on the ultrasound beam produced by the transducer elements and the reflections received by the transducer elements. A control unit may be coupled to the matrix array, e.g., via a cable, for controlling the transducer elements to generate various planar and volumetric ultrasonic beams. Optionally, a foot switch is coupled to the control unit for enabling control of the transducer elements via the control unit.

The invention, together with further objects and advantages thereof, may best be understood by reference to the following description taken in conjunction with the accompanying drawings, wherein like reference numerals identify like elements and wherein:

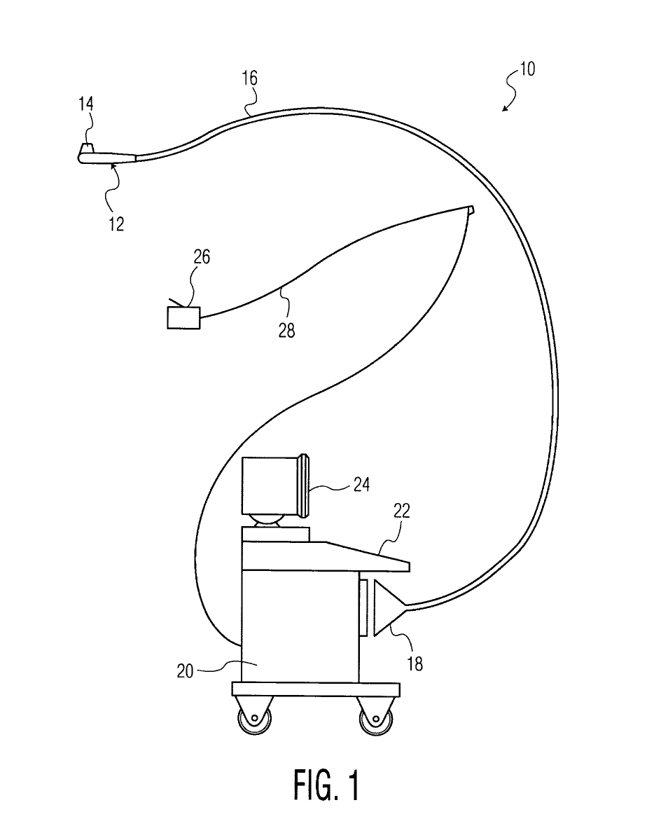

FIG. 1 shows an ultrasound finger probe system in accordance with the present invention.

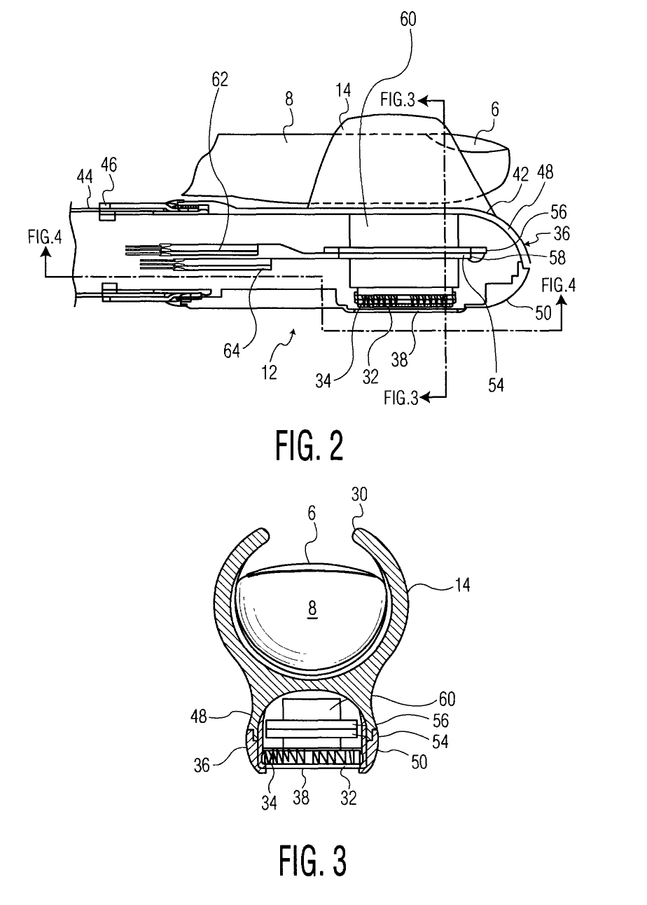

FIG. 2 is a side view of the ultrasound finger probe in accordance with the invention during use.

FIG. 3 is a cross-sectional view of the ultrasound finger probe shown in FIG. 2 taken along the line 3-3 of FIG. 2.

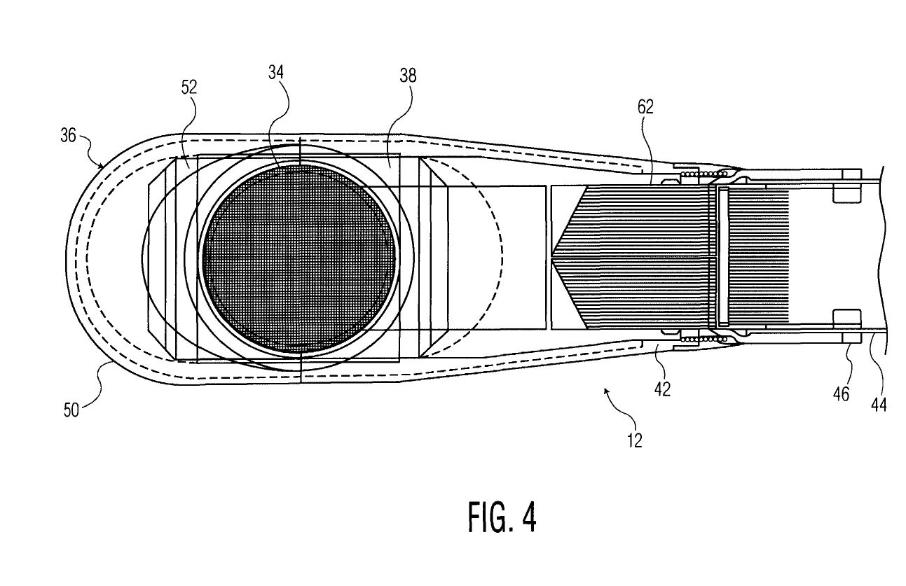

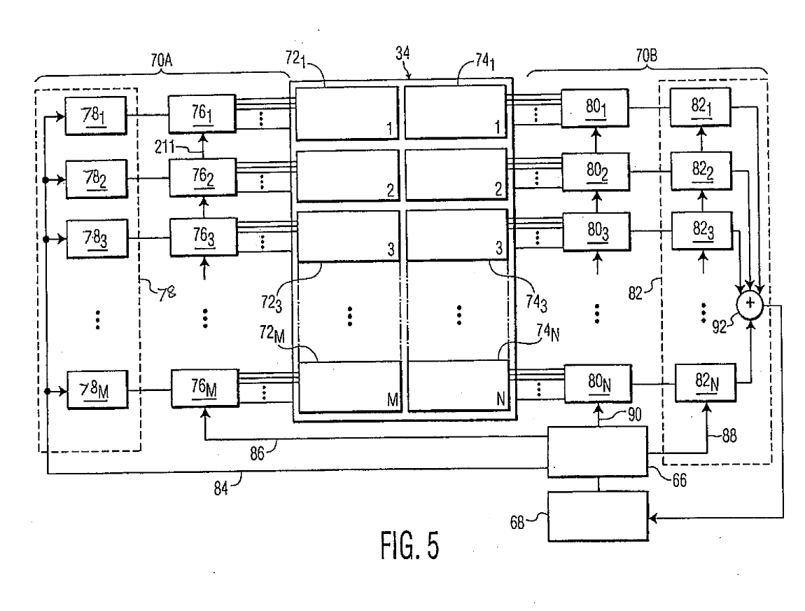

FIG. 4 is a cross-sectional view of the ultrasound finger probe shown in FIG. 2 taken along the line 4-4 of FIG. 2.

FIG. 5 shows diagrammatically an array of ultrasound transducers connected to a transmit beamformer and a receive beamformer of the ultrasound finger probe system in accordance with the invention.

Referring to the accompanying drawings wherein like reference numerals refer to the same or similar elements, FIG. 1 shows an ultrasound probe system 10 of the present invention which includes a finger probe 12 attachable to a finger of a physician or technician by a finger clip 14. Depending on the patient's body part to be viewed, the finger and attached probe 12 are inserted into one of the patient's natural body openings or into a patient's body cavity that has been opened as a result of surgery. For example, during open heart surgery, the functionality of the heart and blood flow in the arteries may be monitored using ultrasound images produced by the ultrasound probe system 10. In addition to intracavity imaging, the ultrasound probe system 10 can also be used externally, i.e., placed over an object of which ultrasound images are desired.

A cable 16 connects the probe 12 to a system control unit 20 through a connector 18. The control unit 20 includes a transmit beamformer unit, a receive beamformer unit and an image generator discussed below with reference to FIG. 5. The control unit 20 is interfaced with a keyboard 22 and provides imaging signals to a video display 24.

A foot switch 26 is connected to the control unit 20 via a control cable 28. The foot switch 28 or alternatively, controls on the keyboard 22 or through another input device, are used to control the imagining element in the probe 12. The foot switch 26 is available as an auxiliary control unit for enabling basic imaging manipulation, such as two-dimensional imaging, image mode selection and imaging depth selection. Its presence allows the ultrasound operator to have his or her hands perform procedures other than imaging control while the foot switch 26 enables imaging control.

FIGS. 2, 3 and 4 show detailed views of the finger probe 12 of the present invention. FIG. 2 shows a side view of an operator's finger 8 inserted through the finger clip 14 of the probe 12. The clip 14 may be fabricated from plastic, rubber or other suitably deformable material. The clip 14 becomes slightly deformed as the operator's finger 8 is inserted into the clip 14. The resistance of the clip 14 to the slight deformation supplies enough pressure to the finger 8 to firmly hold the probe 12 on the finger 8. The clip 14 also preferably has an open top 30, as shown in FIG. 3, to permit quick withdrawal of the finger 8 from the clip 14 in critical situations in which the physician quickly needs to use both hands for another task. The clip 14 may also be provided with a streamlined shape and rounded edges so as to minimize irritation to the body cavity into which probe 12 is inserted.

To provide imaging, a matrix array 32 of independently addressable transducer elements 34 is arranged within a housing 36 and is positioned behind a stationary acoustic window 38 attached to the housing 36 of the probe 12.

In the non-limiting illustrated embodiment, the probe 12 may includes a distal rigid region 42 coupled to a flexible region 44 at a coupling region 46. Distal region 42 includes the housing 36 which encases the matrix array 32, electrical connections and associated electronic elements. Matrix array 32 is preferably a two-dimensional array of independently-addressable ultrasound transducer elements 34 and can have a planar form or curved form.

Housing 36 includes an upper tip housing 48 and a lower tip housing 50 having an opening in which the acoustic window 38 is received and optionally a matching medium located in front of the matrix array 32. Housing 36 may have a convex shape around the window 38. The acoustic window 38 may also include an ultrasonic lens and a metal foil embedded in the lens material. The lens with the metal foil may assist in distributing heat generated during the ultrasound imaging procedure and some have even considered the lens to provide a cooling effect. In addition, the foil in the lens can act as an RF shield if connected to a continuous shield that runs to the system and then is grounded.

As shown in FIG. 4, the acoustic window 38 is substantially circular. However, it is envisioned that other aperture shapes, including square, rectangular and elliptical shapes, can be used for the matrix array 32.

The matrix array 32 may take any form known in the art which provides a plurality of independently-addressable transducer elements enabling an electronically configurable two-dimensional array capable of being controlled to obtain images of an object in multiple planes and in three-dimensions. In one embodiment shown in FIGS. 2-4, the transducer elements 34 of the matrix array 32 are bonded to an array backing 52 and the individual transducer elements 34 are connected to an integrated circuit 54 which is connected to a circuit board 56 using wire bonds 58 or another electronic coupling technique. This structure is thermally connected to a heat sink 60.

Probe 12 also includes two flex circuits 62 and 64, which provide connections between the circuit board 56 and the connector 18, to enable electrical connection between the circuit board 56 and the control unit 20. The super flex circuits 62,64 are arranged to have isotropic bending properties, for example, by folding into an accordion shape or by wrapping into a spiral shape. Alternatively, the super flex circuits 62,64 may be replaced by a coaxial cable or another comparable connecting mechanism for providing electrical connection between the circuit board 56 and the connector 18 associated with the control unit 20.

In a preferred use, the finger 8 is oriented in the clip 14 such that the fingernail 6 is opposite the matrix array 32. Using an electrical signal produced by the control unit 20, the matrix array 32 emits an ultrasound beam. The scanned ultrasound beam defines an image plane the parameters of which are dependent on the electrical signal produced by the control unit 20. The ultrasound beam interacts with the patient's body part and signals are received by the control unit 20 and used to produce an ultrasound image of the patient's body part which is shown on the display 24. The probe 12 can be precisely aimed by the physician using his or her finger 8. Thus, the control unit 20 generates different signals to provide different scanning beams, as known in the art. This type of electronic control of the matrix array 32 is described below. The foot switch 26 can be designed to cause variations in the signals generated by the control unit 20.

In contrast to the prior art finger probes in which mechanical rotation of the imaging element is required to obtain multiple images in a single plane, the electronic control of the matrix array 32 eliminates the need to provide for rotation of any components in the finger probe while still enabling multiple images in a single plane to be obtained, and in addition enables volumetric, three-dimensional images to be obtained.

That is, rotation of the image plane in the finger probe 12 in accordance with the invention is provided electronically, i.e., by appropriate control of the independently-addressable transducer elements 34 in the matrix array 32 via the signals from the control unit 20. Rotation of the image plane is especially desirable in interoperative imaging applications in which various imaging planes can not be accessed by simply changing the rotational orientation of the probe 12 and finger 8 due to space constraints of the imaging environment. Often space constraints limit the maneuverability of the probe 12 once it is inserted into a patient's body. A variety of views of the patient's body part are thus obtainable from a single positioning on the probe 12 of the present invention in conjunction with appropriate control of the imaging via the control unit 20.

Referring now to FIG. 5, the transducer elements 34 in the matrix array 32 are controlled by a control processor 66 housed in the control unit 20. Control processor 66 receives input commands from input controls and provides output control signals. Control processor 66 provides control data to a beamformer, and provides image control data to image generator 68, which includes processing and display electronics, to enable the formation of images on the display 24. The beamformer includes a transmit beamformer 70A and a receive beamformer 70B which may be analog or digital beamformers.

As noted above, the matrix array 32 is a two-dimensional array of ultrasound transducer elements 34 which are arranged into groups of elements (i.e., sub-arrays) using electronically-controllable switches. The switches can selectively connect transducer elements together to form sub-arrays having different geometrical arrangements. That is, the two-dimensional array is electronically configurable. The switches also connect the selected configuration to transmit beamformer 70A or receive beamformer 70B. Each geometrical arrangement of the transducer elements is designed for optimization of the transmitted ultrasound beam or the detected receive beam.

The matrix array 24 includes designated transmit sub-arrays 72₁, 72₂, . . . , 72_{M} and designated receive sub-arrays 74₁, 74₂, . . . , 74_{N}. Transmit sub-arrays 72₁, 72₂, . . . , 72_{M} are connected to intra-group transmit pre-processors 76₁, 76₂, . . . , 76_{M}, respectively, which in turn are connected to transmit beamformer channels 78₁, 78₂, . . . , 78_{M}. Receive sub-arrays 74₁, 74₂, . . . , 74_{N} are connected to intra-group receive pre-processors 80₁, 80₂, . . . , 80_{N}, respectively, which in turn are connected to receive beamformer channels 82₁, 82₂, . . . , 82_{N}. Each intra-group transmit pre-processor 76 may include one or more digital pulse generators that provide the transmit pulses and one or more voltage drivers that amplify the transmit pulses to excite the connected transducer elements. Alternatively, each intra-group transmit pre-processor 76 includes a programmable delay line receiving a signal from a conventional transmit beamformer.

Each intra-group receive pre-processor 80 may include a summing delay line, or several programmable delay elements connected to a summing element (a summing junction). Each intra-group receive processor 80 delays the individual transducer signals, adds the delayed signals, and provides the summed signal to one receive beamformer channel 82. Alternatively, one intra-group receive processor provides the summed signal to several receive beamformer channels 82 of a parallel receive beamformer. The parallel receive beamformer is constructed to synthesize several receive beams simultaneously. Each intra-group receive pre-processor 80 may also include several summing delay lines (or groups of programmable delay elements with each group connected to a summing junction) for receiving signals from several points simultaneously.

Control processor 66 provides delay commands to transmit beamformer channels 78₁, 78₂, . . . , 78_{M} via a bus 84 and also provides delay commands to the intra-group transmit pre-processors 76₁, 76₂, . . . , 76_{M} via a bus 86. The delay data steers and focuses the generated transmit beams over transmit scan lines of a selected transmit pattern. Control processor 66 also provides delay commands to receive beamformer channels 82₁, 82₂, . . . , 82_{N} via a bus 88 and delay commands to the intra-group receive pre-processors 80₁, 80₂, . . . , 80_{N} via a bus 90. The applied relative delays control the steering and focusing of the synthesized receive beams. Each receive beamformer channel 82 may include a variable gain amplifier, which controls gain as a function of received signal depth, and a delay element that delays acoustic data to achieve beam steering and dynamic focusing of the synthesized beam. A summing element 92 receives the outputs from beamformer channels 82₁, 82₂, . . . , 82_{N} and adds the outputs to provide the resulting beamformer signal to image generator 68. The beamformer signal represents one receive ultrasound beam synthesized along one receive scan line.

The matrix array 32 may include a larger number of elements 34 wherein only selected elements are connected to the integrated circuit. Matrix array 32 has the individual transducer elements 34 arranged in rows and columns. The electronically-controllable switches selectively connect the elements in the adjacent rows and columns. Furthermore, the matrix array 32 may also include electronically-controllable switches for selectively connecting adjacent, diagonally-located transducer elements. The selected transducer elements can be connected to the transmit or receive channels of the imaging system. A T/R switch connects the same groups of elements alternatively to the transmit or receive channels. The connections may be direct or may be indirect through one or more other transducer elements.

By appropriately connecting the elements into groups and phasing the elements by the transmit beamformer, the generated ultrasound beam is transmitted along a desired scan line and is focused at a desired depth. The transducer elements may be connected in columns together by closing neighboring column switches. Each column is then connected via one selected transducer element of a selected row to a different system channel, as shown in FIG. 5. The phased transducer elements then form an imaging plane that is perpendicular to the plane of the array and is vertical (i.e., parallel to the selected column).

However, the imaging system can generate the scanned volume by the image planes oriented arbitrarily relative to the transducer rows and having columns. For example, transducer elements in different rows and columns are interconnected to system channels to provide imaging in a plane that is oriented at an angle with respect to the transducer rows and columns. For example, the transducer elements of neighboring rows and columns may be connected to the beamformer in a step-like pattern. This configuration provides the images parallel to a plane that is oriented at about 45 degrees with respect to the column orientation.

In another embodiment, the transducer elements may be connected to the beamformer to form approximately circular contours. This improves the elevation focus control. The acoustic center can be placed on any element that is connected to a system channel. In general, the transducer configurations can be combined with the elevation focus control by determining the appropriate equal delay contours and connecting elements along those contours.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1.-6. (canceled)

6. A system for ultrasonically interrogating a patient's body part and for producing ultrasound images based on the interrogation, the system comprising:
an ultrasound probe including a matrix array arranged to produce an ultrasound beam and receive reflections of the beam by the patient's body part, said matrix array comprising a two-dimensional array of independently-addressable transducer elements controlled such that ultrasound beams are transmitted along desired scanlines to obtain images of the body part in multiple planes and in three dimensions;
a finger clip coupled to said probe to enable attachment of said probe to an operator's finger;
a system control unit comprising a beamformer unit and an image generator;
a cable which connects the probe to the system control unit; and
a display unit coupled to the image generator for displaying ultrasound images based on the ultrasound beams produced by said transducer elements and the reflections received by said transducer elements from the multiple planes and three dimensions of the body part.

7. The system of claim 6, wherein the control unit is coupled to said matrix array for controlling said transducer elements to generate various planar and volumetric ultrasonic beams.

8. (canceled)

9. (canceled)

10. The system of claim 7, further comprising a foot switch coupled to said control unit for enabling control of said transducer elements via said control unit.

11. The system of claim 6, wherein said probe includes a housing, an array backing arranged in said housing, a circuit board arranged in said housing and an integrated circuit arranged in said housing and connected to said circuit board, said transducer elements being bonded to said array backing and connected to said integrated circuit.

12. The system of claim 6, wherein said transducer elements of said matrix array are segmented into transmit sub-arrays and receive sub-arrays, each of said transmit sub-arrays being connected to a respective intra-group transmit pre-processor which is connected to a respective transmit beamformer channel, each of said receive sub-arrays being are connected to a respective intra-group receive pre-processor which is connected to a respective receive beamformer channel.

13. The system of claim 6, wherein said probe includes a housing, said matrix array being arranged in said housing and being non-rotatable relative to said housing.

14. A method for ultrasonically interrogating a patient's body part and for producing ultrasound images based on the interrogation, comprising the steps of:
arranging a matrix array in a housing of an ultrasound probe, the matrix array comprising a non-rotatable two-dimensional array of independently-addressable transducer elements;
coupling a finger clip coupled to the housing of the probe to enable attachment of the probe to an operator's finger;
placing the housing of the probe over the patient's body part;
electronically controlling the transducer elements to produce an ultrasound beam and receive reflections of the beam from multiple planes and three dimensions of the patient's body part;
coupling the matrix array to a control unit comprising a beamformer and an image generator; and
displaying ultrasound images generated from the reflections received by the transducer elements.

15. The method of claim 14, wherein the transducer elements are controlled by the control unit coupled to the matrix array.

16. The method of claim 15, further comprising the steps of:
coupling a foot switch to the control unit; and
providing imaging control devices on the foot switch to enable control of the transducer elements via the control unit.

17. The method of claim 14, further comprising the steps of:
segmenting the transducer elements into transmit sub-arrays and receive sub-arrays;
connecting each of the transmit sub-arrays to a respective intra-group transmit pre-processor which is connected to a respective transmit beamformer channel; and
connecting each of the receive sub-arrays being to a respective intra-group receive pre-processor which is connected to a respective receive beamformer channel.
